(19) United States
(12) Patent Application Publication
(10) Pub. No.: US 2013/0123625 A1
(43) Pub. Date: May 16, 2013

(54) INTEGRATED ULTRASOUND ABLATION AND IMAGING DEVICE AND RELATED METHODS

(71) Applicant: BOSTON SCIENTIFIC SCIMED, INC., MAPLE GROVE, MN (US)

(72) Inventors: ROGER N. HASTINGS, MAPLE GROVE, MN (US); MICHAEL J. PIKUS, GOLDEN VALLEY, MN (US); KEVIN D. EDMUNDS, HAM LAKE, MN (US); SCOTT R. SMITH, CHASKA, MN (US); LEONARD B. RICHARDSON, BROOKLYN PARK, MN (US); TAT-JIN TEO, SUNNYVALE, CA (US)

(73) Assignee: BOSTON SCIENTIFIC SCIMED, INC., MAPLE GROVE, MN (US)

(21) Appl. No.: 13/676,871

(22) Filed: Nov. 14, 2012

Related U.S. Application Data
(60) Provisional application No. 61/559,491, filed on Nov. 14, 2011.

(51) Int. Cl. A61N 7/00; A61B 8/12

(52) U.S. Cl. CPC A61N 7/00; A61B 8/12; A61N 2007/0052; A61N 2007/003
USPC 600/439

(57) ABSTRACT

Intravascular nerve modulation systems and methods for making and using the same are disclosed. An example system may include an elongate shaft having a proximal end region and a distal end region and a central longitudinal axis. An ablation transducer may be disposed at the distal end region. The system may also include a rotational drive configured to rotate the ablation transducer about the central longitudinal axis. A control and power system may be operably connected to the ablation transducer and the rotational drive. In some instances, the ablation transducer may be a combined ablation and imaging transducer.

### CROSS-REFERENCE TO RELATED APPLICATIONS

This application claims priority under 35 U.S.C. §119 to U.S. Provisional Application Ser. No. 61/559,491, filed Nov. 14, 2011, the entirety of which is incorporated herein by reference.

### TECHNICAL FIELD

The present invention relates to methods and apparatuses for nerve modulation techniques such as ablation of nerve tissue or other destructive modulation technique through the walls of blood vessels and monitoring thereof.

### BACKGROUND

Certain treatments require the temporary or permanent interruption or modification of select nerve function. One example treatment is renal nerve ablation which is sometimes used to treat hypertension or other conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate or otherwise modulate perivascular renal nerves. It is therefore desirable to provide for alternative systems and methods for intravascular nerve modulation.

### SUMMARY

The disclosure is directed to several alternative designs, materials and methods of manufacturing medical device structures and assemblies for performing and monitoring tissue changes.

Accordingly, one example embodiment is a system for nerve modulation that may include an elongate shaft having a proximal end region and a distal end region. A combined ablation and imaging transducer may be disposed at the distal end region and may be connected to the shaft by a motor. The combined ablation and imaging transducer may include one or more unidirectional ablation transducers and one or more imaging transducers operatively connected to a power and control system. The one or more ablation transducers may be concentrically arranged around the one or more imaging transducers. The ablation and imaging transducers of the combined ablation and imaging transducer face in a common direction that is perpendicular to the longitudinal axis of the system and may be rotated by the motor. For the embodiments that include a plurality of ablation transducers, the ablation transducers may be operated as a phased array to focus the ablation energy.

In another example embodiment, a combined ablation and imaging transducer includes a unidirectional ablation transducer or a plurality or array of unidirectional ablation transducers facing in a common direction and an imaging transducer facing in the opposite direction and joined to the back of the ablation transducers.

In another, example embodiment, a device as described above is inserted into a patient to a desired treatment area. The distal portion of the device is rotated and the imaging transducer is activated to provide a cross-sectional image of the treatment area. The ablation transducers are activated to ablate the target tissue, and the energy is adjusted manually or automatically by the system to tailor the ablation treatment to particular angular and radial locations of the treatment area.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ;

FIG. 2 illustrates a distal end of an illustrative renal nerve modulation system;

FIG. 3A illustrates an example combined ablation and imaging transducer;

FIG. 3B illustrates a cross-sectional view of the transducer of FIG. 3A;

FIG. 4 illustrates a cross-sectional view of another example combined ablation and imaging transducer;

FIG. 5A illustrates another example combined ablation and imaging transducer; and

FIG. 5B illustrates a cross-sectional view of the transducer of FIG. 5A.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. For example, the devices and methods described herein may also be used for prostate ablation, tumor ablation, and/or other therapies requiring heating or ablation of target tissue. In some instances, it may be desirable to ablate perivascular renal nerves with deep target tissue heating. As energy passes from a modulation element to the desired treatment region the energy may heat the fluid (e.g. blood) and tissue as it passes. As more energy is used, higher temperatures in the desired treatment region may be achieved thus resulting in a deeper lesion. Monitoring tissue properties may, for example, verify effective ablation, improve safety, and optimize treatment time.

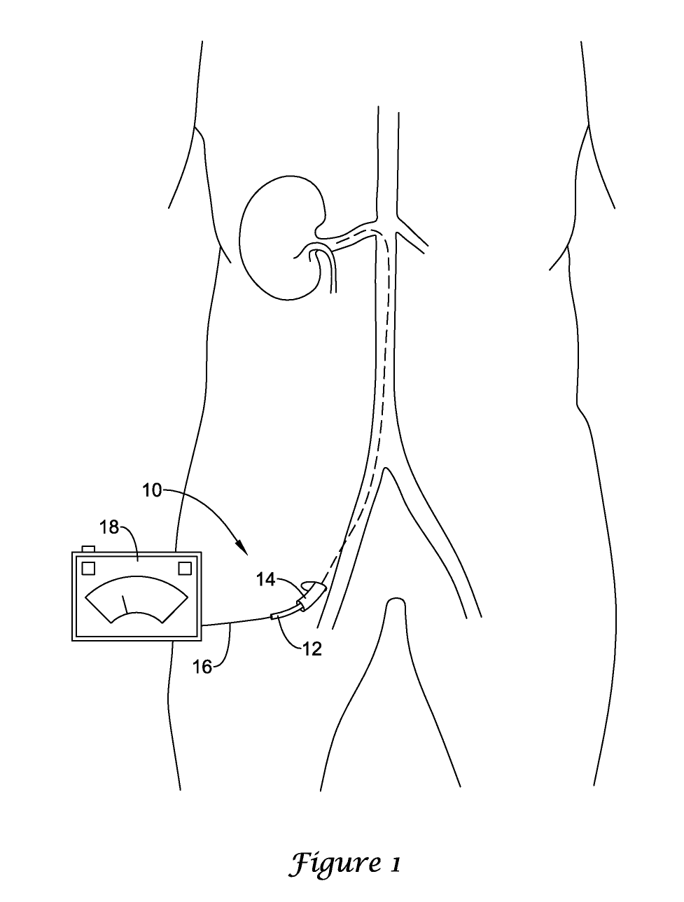

FIG. 1 is a schematic view of an illustrative renal nerve modulation system 10 in situ. System 10 includes an imaging and modulation catheter 12 that may be disposed in an optional sheath or guide catheter 14. Imaging and modulation catheter 12 may be connected through an element 16 to a control and power system 18, which supplies the necessary electrical energy to activate the one or more modulation elements at or near a distal end of the catheter 12. The control and power system 18 may include monitoring elements to monitor parameters such as power, temperature, voltage, pulse size, and/or shape and other suitable parameters as well as suitable controls for performing the desired procedure. It is contemplated that more than one control and power system 18 may be provided as desired or that the control and power element, illustrated as a single package may in some embodiments be multiple elements. For example, in some embodiments, there may be a separate monitoring or display element and a separate control or power element. It can be appreciated that this is a simplified view of a system that may include many other elements. For example, the system 10 may include one or more proximal hubs each having one or more ports such as flush ports. One hub may be connected to guide catheter 14 and another hub may be connected to imaging and modulation catheter 12.

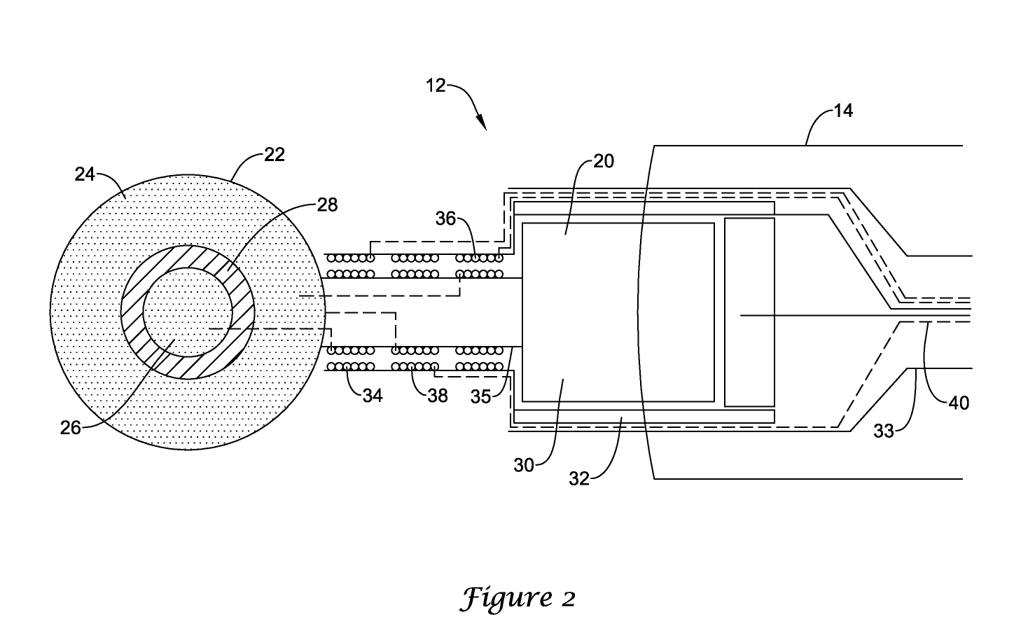

FIG. 2 illustrates a distal end of an imaging and modulation catheter 12 of an illustrative renal nerve modulation system disposed in a sheath or guide catheter 14. This system includes a motor 20 connected to a combined imaging and ablation transducer 22. The motor may be operated to rotate the combined imaging and ablation transducer 22 relative to the proximal portion of the catheter. The motor 20 and associated controller may allow for a constant preselected rotational velocity of, for example, 2, 3, 4, 5, 10, 20, 30, 40 or more Hz (i.e. rotations/second) or may allow for an adjustable rotational velocity of between 0 and 50 Hz, between 0 and 20 Hz, between 0 and 10 Hz, between 0 and 5 Hz, between 0 and 4 Hz, or other desired ranges. In some embodiments, the motor 20 is a micro-motor or stepper motor that can rotate the combined imaging and ablation transducer 22 to a desired angular position. Based on the received user input, the control and power element 18 determines an appropriate signal, such as one or more voltages, to send to motor 20 to move the combined imaging and ablation transducer 22 to the desired angular position. In some embodiments, the motor 20 and associated controls can perform, as desired and as based on received input or preset programs, any of the functions of a constant rotational velocity, an adjustable or variable angular velocity and rotating the combined imaging and ablation transducer 22 to a particular angular position. It can be appreciated that the angular resolution of the control may depend upon the particular motor. For example, a particular motor may be able to move the combined imaging and ablation transducer 22 to one of 100 angular positions that are spaced at 3.6 degrees from each other.

As illustrated in FIG. 2, the combined imaging and ablation transducer 22 includes an imaging transducer 26 separated from an ablation transducer 24 by insulation 28. The combined imaging and ablation transducer 22 may be mechanically connected to a rotor 30 or other rotational element of motor 20 by a shaft 35 or other suitable means and may be electrically connected to the control and power system 18 through transformers 34, 36 and 38 and suitable leads 40. The stator 32 or other stationary portion of motor 20 may be connected to a shaft 33 that extends back to the proximal portions of the system. The stator 32 is electrically connected to the control and power system 18 through suitable leads 40. It can be appreciated that in some embodiments, other elements such as slip rings, commutators or brushes may be substituted for transformers 34, 36 and 38.

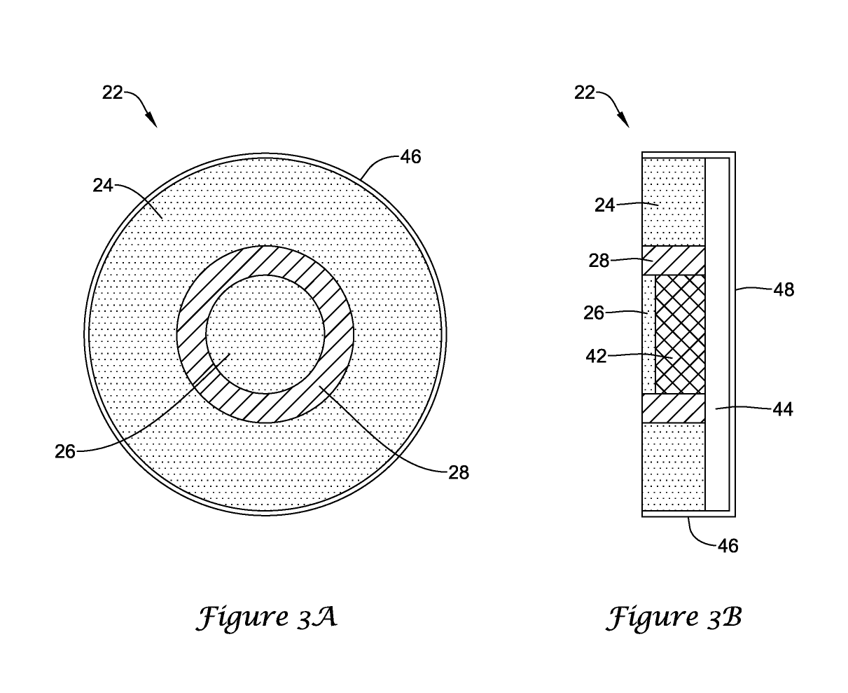

FIGS. 3A and 3B illustrate a combined imaging and ablation transducer 22 in more detail. The combined imaging and ablation transducer 22 includes an imaging transducer 26 separated from an ablation transducer 24 by insulation 28. The transducers are typically made from a piezoelectric material such as PZT (lead zirconate titinate) sandwiched between two conductive layers. When a voltage is introduced across the material, the material thickness and thins with the voltage. If the voltage is applied in the form of a sine wave, the material will vibrate in tune with the voltage. The thickness of the ablation transducer 24 may be selected to have a resonance frequency that corresponds to the preferred frequency to be used for ablation. A typical thickness may correspond to half of the wavelength of the preferred frequency. The ablation frequency is selected for a desired depth of penetration into the tissue; the depth of effective penetration is inversely related to the frequency. For example, one contemplated ablation frequency is 20 MHz although other ablation frequencies in the range of 5 MHz to 20 Hz or more may be chosen. At this frequency, the ablation energy will penetrate about 4 mm. Any frequency may be suitable depending on the application. In some renal nerve ablation applications, a frequency that reaches a depth of about 0.5 to 3.0 mm may be suitable. In some embodiments, the frequency may be adjusted during use to vary the depth of penetration by the control and power system 18. The level of ablation energy is usually selected to keep the temperature of the affected tissue below 65° C. In some embodiments, a range of 55-65° C. is appropriate. In some embodiments a range of about 50° C. is appropriate. For an ablation transducer of about 1.5 mm in diameter, the power may be applied in a range of 1-8 watts, a small portion of which is lost as heat in the blood stream while the major portion enters the tissue wall.

The imaging transducer 26 operates by sending short pulses of ultrasonic energy that are reflected back and measured by the imaging transducer. A piezoelectric material produces a voltage when it is compressed and these voltages are measured and recorded to provide an image of the tissue layer near the vessel wall. An example frequency used for imaging is 40 MHz although any frequency in the range of 20 MHz to 80 MHz may be used. A suitable interval for the pulse may be one wavelength of the frequency. The interval between the pulses should be long enough to allow the echoes from the pulse to return from the deepest tissue being imaged. The imaging transducer is backed by a damping layer 42 that absorbs ultrasound energy to preventing ringing of the imaging transducer. A suitable material for the damping layer 42 may be a tungsten-filled epoxy or the like.

The imaging and ablation transducers may also include a matching layer (not illustrated) on the side configured to deliver the ultrasound energy. For a transducer configured to deliver ultrasound energy on two sides, there may be a matching layer on both of the sides. The matching layer provides acoustic impedance matching to increase the efficiency of the energy transmission. The matching layer may be selected such that the acoustic impedance of the matching layer is equal to the geometric mean of the transducer crystal material and the adjacent media (e.g. blood). A suitable matching layer may be a silver-filled epoxy. The thickness of the matching layer is typically one-quarter of the length of a wavelength of the desired output frequency.

For a transducer configured to deliver ultrasonic energy on only one side, a backing layer 44 is typically included on the non-ultrasonic energy emitting side. Backing layer 44 is typically a vacuum or air filled cavity that forms an acoustic barrier. The backing layer 44 may be formed by securing the transducer in an impervious holder having a side 46 and back 48 to create the air cavity or vacuum of the backing layer 44. The transducer may be secured by using a flexible adhesive such as silicone.

In some contemplated embodiments, the combined imaging and ablation transducer 22 may include a two-side ablation transducer 24. In such embodiments, the backing layer 44 is omitted and a second matching layer (not illustrated) may be included instead. The backing layer 44 may be omitted by fixing the transducer in a holder that omits back 48 or by omitting the holder altogether. The imaging transducer, having damping layer 42, will continue to operate in only one direction.

A combined imaging and ablation transducer 22 includes conductive layers on both flat surfaces of both transducer 24 and 26, as discussed above. These conductive layers may include a tie layer, such as, for example, a layer of chrome directly on the surface of the crystal and then a layer of gold, or a layer of chrome, a layer of nickel and then a layer of gold. The conductive layers and tie layer may be vapor deposited, sputtered, electroplated or the like onto the surfaces of the crystals. These conductive surfaces are electrically connected to leads 40. In some embodiments, the fronts of transducers 24 and 26 are preferably connected by separate leads, while the backs of transducers 24 and 26 may share a common return lead.

In this embodiment, combined ablation and imaging transducer 22 is disc-shaped, and ablation transducer 24 forms the outer part of the disc. Ablation transducer 24 defines a cavity in which imaging transducer 26 is secured. Imaging transducer 26 is separated from ablation transducer 24 by an insulator 28. Insulator 28 provides electrical and acoustical isolation between the two transducers 24, 26. The combined ablation and image transducer 22 is, in some applications, sized to fit in a 6 French guide catheter, and may have a diameter of approximately 1.5 mm. In some applications, the combined ablation and image transducer 22 has an elongate shape. It may, for example, be a rectangle having a width of 1.5 mm and a length of 4 mm.

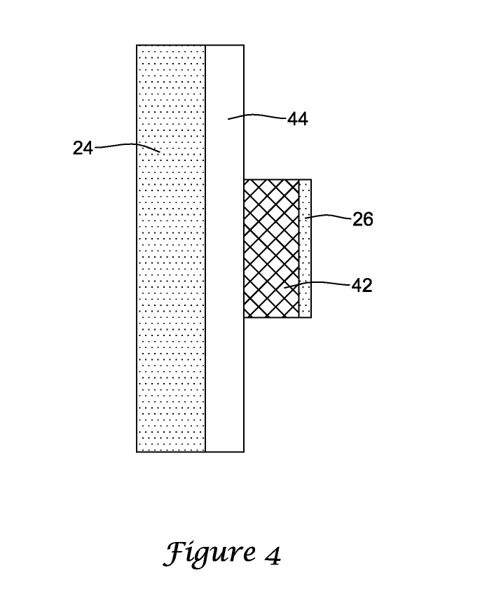

Many other configurations of combined ablation and imaging transducer 22 are contemplated. For example, FIG. 4 is a side view of another combined ablation and imaging transducer, where the ablation transducer 24 and the imaging transducer 26 are back to back. In this embodiment, ablation transducer 24 may be disc-shaped, and need not have the hole to receive the imaging transducer 26. A backing layer 44 may be provided to direct the ablation energy in one direction (or may be omitted to provide for bi-directional ablation). The imaging transducer preferably includes damping layer 42. Both transducers also may include matching layers as described above. One variation of this configuration is to change the shape of the transducers into oblongs or rectangles as described above.

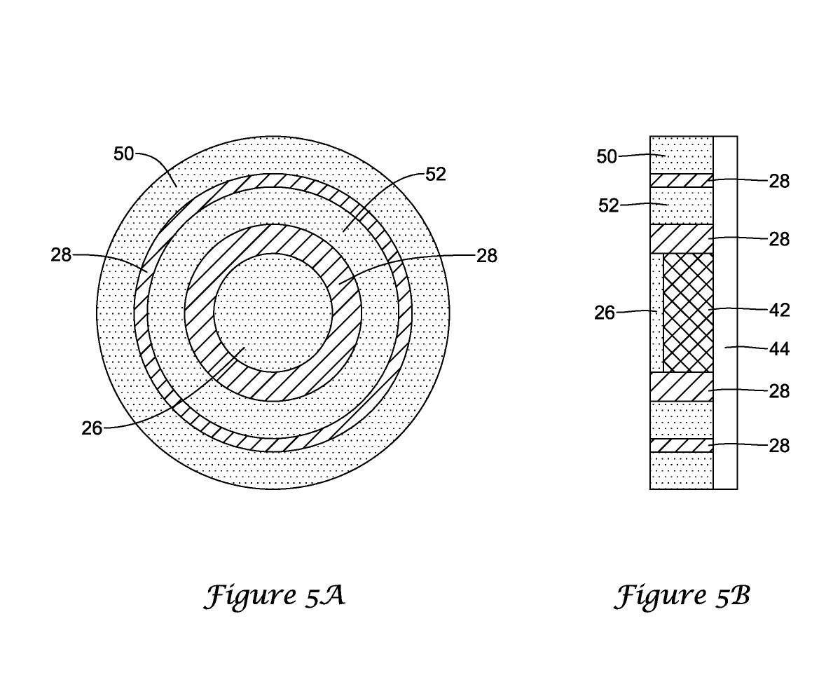

FIG. 5 illustrates another example configuration. This combined ablation and imaging transducer includes a first ablation transducer 50 surrounding a second ablation transducer 52, which, in turn, surrounds an imaging transducer 26. The three transducers are separated by insulating layers 28 and are electrically connected to the control and power system so as to allow the separate activation of each transducer. The energy produced from a transducer extends in a generally collimated fashion. The two transducers 50, 52 allow for the control and power system to focus the ablation energy by, for example, varying the phases of the power sent to the two transducers, 50, 52, respectively. Further or more refined focusing may be achieved by including a third or a third and a fourth ring ablation transducer, with the associated power and control circuitry. Lateral focusing may be achieved by dividing one or more of the rings into two, three, four or more separately controlled segments. A rectangular ablation and imaging transducer could be used for focusing by providing a rectangular array of ablation transducers. Changing the phase may allow various depths of tissue to be selectively ablated.

In other contemplated embodiments, the transducers may double as ablation and imaging transducers. The transducers may have a modest damping layer to supply enough damping for imaging but little enough to generate adequate power for ablation. Imaging at frequencies higher than the ablation frequency may be accomplished by delivering higher energy image pulses at a higher frequency that is still in the transducer bandwidth.

Further, other embodiments where the ablation or nerve modulation element is something other than an ultrasonic transducer are also contemplated. For example, in some embodiments, one or more radio-frequency ablation electrodes may be substituted for ablation transducer 24. Other contemplated ablation technologies include cryogenic ablation and laser ablation.

It can be appreciated that any of the described embodiments of imaging and control transducers may be readily combined with systems such as that of FIG. 2 with minor modifications such as the introduction of further transformers and appropriate adjustments to the control and power system.

The system of FIG. 2 includes the combined ablation and imaging transducer 22 attached to a motor 20 at the distal end of a shaft 33. In other contemplated embodiments, the motor 20 and shaft 33 may be replaced by a drive shaft powered by an external motor or actuator. In still other contemplated embodiments, the system does not include a motor, drive shaft or the like, but rather is hand-operated.

Any of the contemplated embodiments may include further elements, such as radiopaque elements for visualization, an echo-lucent and/or radio-lucent sheath that surrounds the combined ablation and imaging transducer (e.g., which may include an echo-lucent guide catheter 14), a focusing balloon disposed on or around the combined ablation and imaging transducer that contains a fluid with a different specific gravity than the blood, fins or impellers that may help to direct blood flow over the combined ablation and imaging transducer and thereby aid in cooling the combined ablation and imaging transducer, one or more centering balloons or struts, and so forth. The disclosure is therefore intended to be read expansively.

One example use will be described with reference to the embodiment of FIG. 2 having the single direction ablation transducer of FIGS. 3A and 3B. The catheter 12 may be introduced to a desired location through a guide catheter 14 or through another suitable method. The device may be moved to the desired location along with the guide catheter 14, and then the guide catheter 14 may be withdrawn proximally to expose the distal end of the device or the device may be advanced distally out from the distal end of the sheath. One contemplated use is the ablation or modulation of renal nerves proximate the wall of a renal artery. Therefore, the distal region of the catheter 12 may be introduced into a renal artery. The combined ablation and imaging transducer 22 may be rotated with only the imaging transducer providing an initial cross-sectional image of a contemplated treatment area. If the treatment area is found suitable, the procedure may continue; if not, the device may be moved to another potential treatment area. In one contemplated alternative, the device is rotated as it is pulled back through the renal artery to create an initial 3D image along a length of the renal artery. The physician may then select a position that is best suited for the treatment. One reason a treatment area may be deemed unsuitable is the presence of calcium deposits. The rotation may be a constant rotation powered by the motor 20 and may be at 3 Hz, 10 Hz, 20 Hz, 30 Hz, 40 Hz or other desired speed. The initial imaging may also provide a baseline image of the treatment area prior to activating the ablation transducer 24.

The physician may then start a program that activates the ablation transducer 24. The ablation transducer 24 may be activated simultaneously with the imaging transducer, or alternately. As the treatment proceeds, the ultrasound image is monitored by the physician to observe the change in the tissue caused by ablation as the procedure progresses. The physician can then adjust the procedure by reducing (or increasing) the ablation energy to a particular location. As the combined ablation and imaging transducer is rotated, it is able to deliver a desired level of ablation energy to a particular angular or radial location, thus adjusting the ablation treatment based on feedback from the imaging transducer. This may be done through a graphical user interface on the power and control system or through another suitable method. In another embodiment, the computer automatically analyzes the data from the imaging transducer to sense tissue change and automatically reduce power to ablated tissue. In another embodiment, the physician can rotate the combined ablation and imaging transducer to a specific angular location, and hold it in place while delivering ablation energy to that location.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. An intravascular nerve modulation system, comprising:
an elongate shaft having a proximal end region and a distal end region and a central longitudinal axis;
an ablation transducer disposed at the distal end region;
a rotational drive configured to rotate the ablation transducer about the central longitudinal axis; and
a control and power system operably connected to the ablation transducer and the rotational drive.

2. The intravascular nerve modulation system of claim 1, wherein the ablation transducer is a combined ablation and imaging transducer.

3. The intravascular nerve modulation system of claim 1, wherein the ablation transducer comprises a single planar ablation transducer.

4. The intravascular nerve modulation system of claim 1, wherein the ablation transducer comprises a plurality of ablation transducers, the plurality of ablation transducers disposed in a single plane.

5. The intravascular nerve modulation system of claim 1, wherein the ablation transducer comprises a first ablation transducer and a second ablation transducer, the second ablation transducer defining a first cavity in which the first ablation transducer is disposed.

6. The intravascular nerve modulation system of claim 5, wherein the ablation transducer further comprises a third ablation transducer, the third ablation transducer defining a second cavity in which the second ablation transducer is disposed.

7. The intravascular nerve modulation system of claim 1, wherein the ablation transducer is configured to send an ablation pulse in a first direction normal to the central longitudinal axis.

8. The intravascular nerve modulation system of claim 1, wherein the ablation transducer is configured to send an ablation pulse in only a single direction at a given time.

9. The intravascular nerve modulation system of claim 1, wherein the ablation transducer is configured to send an ablation pulse in a first direction and a second direction opposite the first direction.

10. A nerve modulation system, comprising:
an elongate shaft having a proximal end region and a distal end region and a central longitudinal axis;
a combined transducer disposed at the distal end region, the combined transducer including an ablation transducer and an imaging transducer;
a rotational drive configured to rotate the combined transducer about the central longitudinal axis; and
a control and power system operably connected to the combined transducer and the rotational drive.

11. The nerve modulation system of claim 10, wherein the ablation transducer has a front surface, wherein the imaging transducer has a front surface, and wherein the front surfaces of the ablation transducer and the imaging transducers lie in the same plane.

12. The nerve modulation system of claim 10, wherein the ablation transducer defines a cavity and wherein the imaging transducer is disposed in the cavity.

13. The nerve modulation system of claim 10, wherein the combined transducer is configured to send a first ablation pulse in a first direction normal to the central longitudinal axis.

14. The nerve modulation system of claim 13, wherein the combined transducer is configured to send a second ablation pulse in a second direction opposite the first direction.

15. The nerve modulation system of claim 13, wherein the combined transducer is configured to send an ablation pulse in only a single direction at a given time.

16. The nerve modulation system of claim 13, wherein the combined transducer has a rectangular face having a long side and a short side, wherein the short side is between 1.4 and 1.6 mm, and wherein the long side is between 3.7 and 4.5 mm.

17. The nerve modulation system of claim 10, wherein the rotational drive includes a motor disposed between the elongate shaft and the combined transducer.

18. The nerve modulation system of claim 10, further comprising an echo-lucent sheath disposed over the combined transducer.

19. The nerve modulation system of claim 10, further comprising rotating fins to direct cooling blood over the combined transducer.

20. A method of performing a tissue modification procedure, the method comprising:
a) providing a medical device having a distal end region and have at the distal end region an ultrasonic imaging transducer and a tissue modification element;
b) inserting the medical device into a patient's body;
c) activating the ultrasonic imaging transducer to receive data about tissue proximate the ultrasonic imaging transducer; and
d) activating the tissue modification element.
